(19) United States
(12) Patent Application Publication
(10) Pub. No.: US 2026/0273169 A1
(43) Pub. Date: Sep. 17, 2026

(54) MODULE LOCATION REPORTING

(71) Applicant: CareFusion 303, Inc., San Diego, CA (US)

(72) Inventors: Michael K. WORKMAN, Carlsbad, CA (US); Christopher M. DILEO, San Diego, CA (US)

(21) Appl. No.: 19/080,581

(22) Filed: Mar. 14, 2025

(51) Int. Cl. A61M 5/14

(52) U.S. Cl. CPC A61M 5/1408; A61M 2205/14

(57) ABSTRACT

Certain aspects of the disclosure provide systems and methods for detecting coupling of multiple infusion devices and determining a connection position of the coupled infusion devices relative to one another. Coupling data may be used to indicate usage, or overuse, of the connection position. Such usage information may be used to determine when the infusion device is ready for maintenance, cleaning, repair, and/or maintenance. Further, such usage information may be used to generate a configuration recommendation to balance and distribute usage across various connection positions of the infusion device.

### BACKGROUND

### Field

Aspects of the present disclosure relate to systems and methods for modular infusion device monitoring, such as for maintenance and configuration recommendations.

### Description of Related Art

An infusion pump is a type of medical device configured to infuse therapeutic fluid into a patient, such as in a hospital or other medical care site. Generally, an infusion pump is programmed to pump fluid at a specific flow rate and up to a specific volume in order to administer a specific dose of a treatment. In particular, the infusion pump may be configured to receive one or more operational parameters used to control operation of the infusion pump from one or more sources. The one or more operational parameters may include one or more of flow rate, volume, velocity, infusion time, and/or the like. The infusion pump then operates according to the one or more operational parameters.

In some cases, an infusion pump may be modular, for example, part of a system of one or more infusion devices. For example, one or more infusion pumps may be coupled to a patient care unit (PCU) or patient care device (PCD) to control operation of the infusion pump(s). A PCU or PCD may also function as a docking station to couple multiple pumps or other devices together. In addition, a PCU or PCD may couple various ancillary medical devices such as a vital signs monitor, a medication dispensing device (e.g., cabinet, tote), a medication preparation device, an automated dispensing device, or other similar devices. Together, the coupled infusion devices may be referred to as an infusion system. For example, a typical infusion system may include at least one infusion pump and a control device (e.g., a PCD or PCU). However, various combinations or configurations are possible. In one example, four infusion pumps and one PCU may be coupled together as an infusion system. In another example, two infusion pumps and one PCU may be coupled together as an infusion system.

### SUMMARY

Certain aspects provide a method, comprising detecting a coupling of a first infusion device and a second infusion device; storing data indicative of a coupling event based on detecting the coupling of the first infusion device and the second infusion device; determining a recommended connection position of a set of connection positions associated with the second infusion device based on the coupling event; and outputting an indication of the recommended connection position of the set of connection positions for the coupling of the first infusion device and the second infusion device.

Certain aspects provide a method, comprising: detecting a coupling of a first infusion device and a second infusion device; determining a connection position of a set of connection positions associated with the second infusion device based on the coupling of the first infusion device and the second infusion device; and transmitting, to a remote device, a connection message including an identifier of the first infusion device; an identifier of the second infusion device; and an identifier of the connection position of the set of connection positions.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1A depicts an example infusion system.

FIG. 1B depicts an example device configuration monitoring system.

FIG. 2A depicts an example workflow for reporting a device configuration.

FIG. 2B depicts another example workflow for reporting a device configuration.

FIG. 3 depicts an example device configuration chart.

FIG. 4 depicts an example workflow for maintaining devices.

FIG. 5 depicts an example workflow for recommending device configurations.

FIG. 6 depicts an example user interface for device maintenance.

FIG. 7 depicts an example user interface for recommending device configurations.

FIG. 8 depicts an example large volume infusion system.

FIG. 9 depicts an example syringe infusion system.

FIG. 10 depicts an example method for recommending device configurations.

FIG. 11 depicts an example method for maintaining devices.

FIG. 12 depicts an example computing device with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for modular infusion device monitoring, such as for maintenance and configuration recommendations.

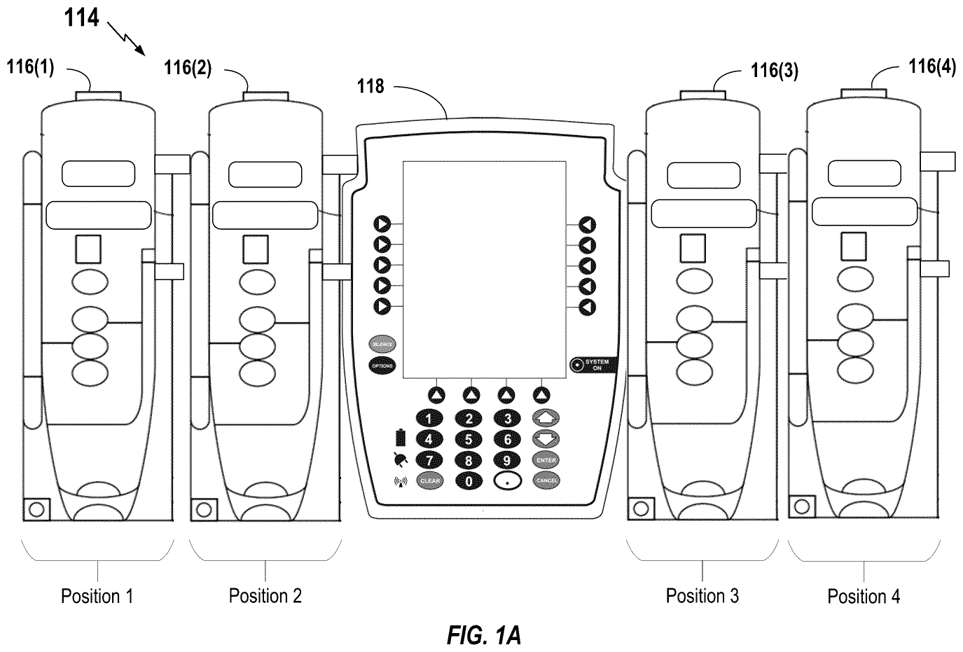

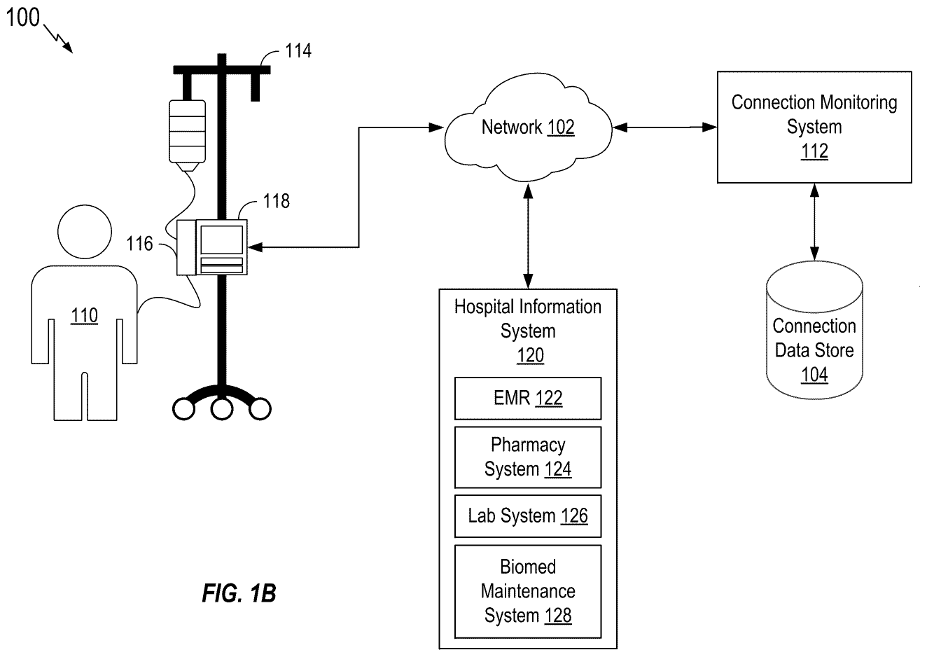

Generally, modular infusion systems may include multiple types of infusion devices coupled together in various configurations. FIG. 1A depicts an example modular infusion system, comprising an infusion pump controller 118 and four infusion pumps, collectively infusion pumps 116. Multiple infusion pumps may be used to facilitate administration of multiple types of fluid to a patient. In some cases, multiple infusion pumps may be used to facilitate administration of a large volume of fluid. In some cases, infusion pumps may be different types, for example, to administer different volumes of fluid, such as a large volume pump and a syringe pump.

During set up of the infusion system, a clinician selects the modules or devices of the infusion system based on the infusion order. That is, based on the type of fluid and dosing information, the clinician selects appropriate devices to configure the infusion system. For example, for a small volume, the clinician may select a syringe pump, while for a large volume, the clinician may select a large volume pump. The clinician also selects a control module, e.g., an infusion pump controller, to control the pump. In some cases, multiple infusion orders may be run for a patient and the clinician selects multiple pumps. A controller may be selected based on the multiple pumps, for example, a controller including a docking station configured for multiple pumps, corresponding to multiple channels.

Generally, the clinician then configures the infusion system using the devices. For example, where there is only one pump and one controller, the clinician may configure the pump in various positions relative to the controller. Environmental factors, personal preference, ease of use, and other considerations may factor into the configuration set by the clinician. For example, where the patient's bed is relative to the infusion system, other medical devices in the area, power supply, or other environmental factors may be considered. In some cases, a clinician may configure the infusion system based on personal preference or ease of use, for example, a right-handed clinician may configure the infusion pump on the right-handed side of the infusion pump controller. Where multiple pumps are to be configured together, the clinician may consider the volume or infusion time of the various infusion orders.

Infusion devices incur wear and tear due to use, including due to attachment and detachment of the devices through connectors. Further, infusion devices may incur wear and tear due to total use time. Because clinicians may configure infusion devices in various configurations, some connectors and/or devices may be overused (e.g., incur additional wear and tear), especially compared to other connectors and/or devices on the same infusion pump controller.

For example, a connector on a right side of the infusion pump controller may incur additional wear and tear due to overuse by right handed clinicians compared to a connector on the left side of the infusion pump controller.

This overuse and increased wear and tear on certain devices and/or connectors may result in additional maintenance, repair, and/or replacement of such devices. Further, uneven wear and tear may shorten usable life of an infusion device.

Additionally, when an infusion device needs maintenance, repair, and/or replacement, the device may be removed from service to obtain the maintenance, before being placed back into service. An infusion device where one connector is overused and incurs additional maintenance, even where other connector(s) have not been overused, may be removed from service for the maintenance, reducing medical care facility supply and additional maintenance.

Aspects of the present disclosure monitor configurations of infusion systems, including for individual devices, to determine connection positions of the infusion devices relative to one another in the infusion system. The connection positions of the infusion devices may further be monitored to determine usage of each connection position, e.g., relative to one another. For example, where an infusion pump is regularly placed on the right side of an infusion pump controller, the right-side connection position may be determined to be overused compared to the left side connection position.

Further, aspects of the present disclosure employ the usage of the various connection positions of the infusion pump controller to determine maintenance for the connection position, e.g., for the connector at that connection position.

Additionally, where one connection position may be overused, or underused, aspects of the present disclosure may generate a configuration to balance the usage of the various connection positions. For example, where an infusion pump is regularly placed on the right side of an infusion pump controller, a generated configuration may indicate to place the infusion pump at the left side of the infusion pump controller to balance the usage. As another example, where a first connector may soon incur maintenance due to usage, a second connector may be suggested in the generated configuration to extend usage of the infusion pump controller.

By generating these configurations, usage and wear and tear of connectors and other devices may be balanced such that usage is distributed across the various connectors. Maintenance of the connectors may then be done for multiple connectors of the infusion device. Time out of service of the infusion device may be reduced, as well as overall maintenance, when multiple connectors are serviced together. Further, extraneous maintenance may be reduced because connectors that are not overused may be indicated as not ready for maintenance yet.

Certain aspects of the present disclosure include detecting coupling of multiple infusion devices and determining a connection position of the coupled infusion devices relative to one another. The connection position may be reported to a monitoring service to record usage. In some aspects, other infusion information may be reported with connection position, for example, start and stop events of the infusion device, attachment and detachment events of the infusion device, total usage time, or the like, which may be used to indicate usage, or overuse, of the connection position.

Certain aspects of the present disclosure may further use such usage information to determine when the infusion device is ready for maintenance, repair, cleaning, replacement, or the like. For example, based on usage information for each connection position, a maintenance event may be determined for the connection position. When a maintenance event is imminent, the infusion device may signal to the clinician to not use the device, place it out of service, or use the device, but place it out of service after the infusion. Thus, overused infusion devices may not be used, and may indicate when ready for service.

Furthermore, certain aspects of the present disclosure may also use such usage information to generate a configuration recommendation to balance and distribute usage across various connection positions of the infusion device. A configuration recommendation may indicate one or more connection positions for connecting the infusion device. In some aspects, a configuration recommendation may be based on infusion information, for example, a pump for a longer infusion may be connected at an inside or lower position relative to the infusion pump controller compared to a pump for a shorter infusion. When the shorter infusion is completed, the infusion pump may be removed without interruption of the pump for the longer infusion. Accordingly, detachment and reattachment may be reduced, as well as fewer interruptions may occur for the longer infusion order.

### Example Intelligent Infusion System

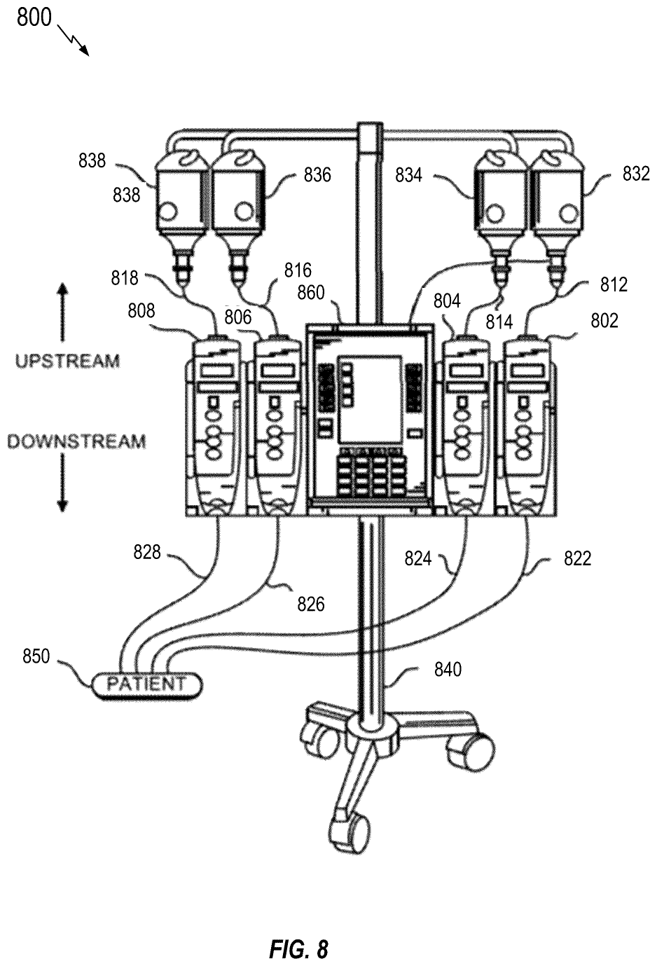

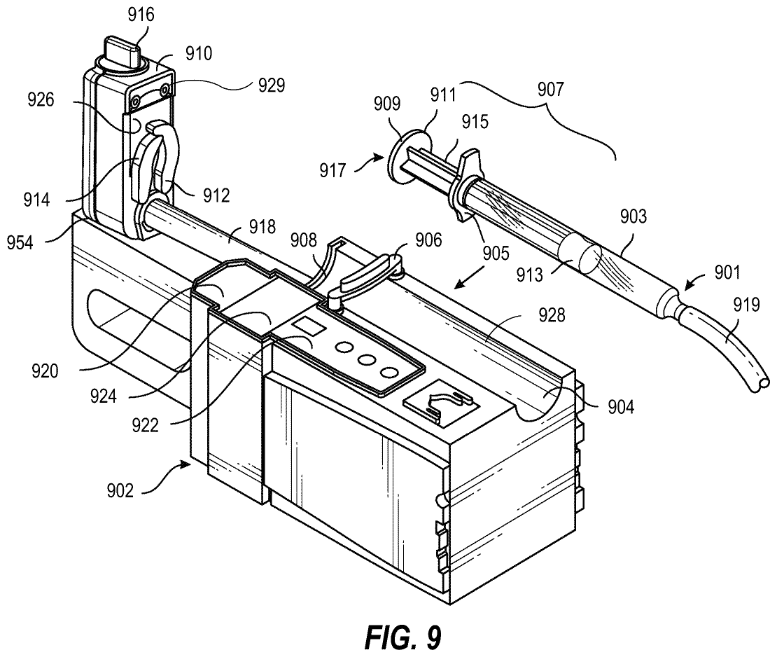

FIG. 1A depicts an example infusion system 114. Infusion system 114 is an electromechanical device for infusing therapeutic fluids into a patient in a controlled and precise manner. Exemplary infusion systems may include a large volume pump or a syringe pump, such as depicted in FIG. 8 or FIG. 9. Specifically, in this example, infusion system 114 includes four large volume pumps, collectively infusion pumps 116. Infusion system 114 comprises an infusion system controller 118 configured to control operations of infusion system 114. Infusion system controller 118 may be an example of a PCU or PCD.

In general operation, therapeutic fluid is loaded into one of the infusion pumps 116, for example, in a syringe, bottle, bag, or other storage container, and the infusion system 114 is configured to pump, or infuse, the therapeutic fluid according to operational parameters of the one infusion pump 116, including, for example, flow rate, volume, and/or time to administer a specific dose of treatment. As described in detail with respect to FIG. 8 or FIG. 9, below, the infusion pump controller 118 and/or the infusion pumps 116 may include one or more buttons, controls, displays, keys, or the like to control operations of the infusion system 114. In some examples, a clinician may utilize the infusion system controller 118 to enter operational parameters or otherwise control operation of the infusion pumps 116. Further, the infusion system controller 118 may display drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers.

In some aspects, the infusion pump controller 118 may contain a memory comprising a drug library or libraries, an event log or logs, and pump configuration settings, such as, but not limited to, profiles to be used in particular care areas such as ICU, PED, etc. The memory may be electronically loadable memory such as non-volatile memory (e.g., EEPROM). Drug libraries stored on infusion systems (which illustratively contain such information as the drug names, ranges of delivery parameter values such as proper concentrations, dosage units, and dose limits) may be used to perform drug calculation-based infusions in a clinical setting. A drug library stored within the infusion system may include clinical order settings such as limits set by the clinical institution for each drug of the library (also referred to as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward (e.g., a care area) of the medical institution in which the drug is being used (for example neonatal care unit (NICU), the intensive care unit (ICU), etc.), and depending on other factors. An alarm may be provided if the clinician sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The depicted infusion system 114 includes four infusion pumps 116 located at four positions. Specifically, a first pump 116(1) is in a first position, a second pump 116(2) is in a second position, a third pump 116(3) is in a third position, and a fourth pump 116(4) is in a fourth position. In some configurations, one pump may be placed in any of the four positions. In some configurations, two pumps may be placed in any two of the four positions, for example, position one and position two, position one and position three, position one and position four, position two and position three, position two and position four, or position three and position four. Similarly, in some configurations, three pumps may be placed in any three of the four positions, for example, positions one, two, and three, positions one, two, and four, positions one, three, and four, or positions two, three, and four.

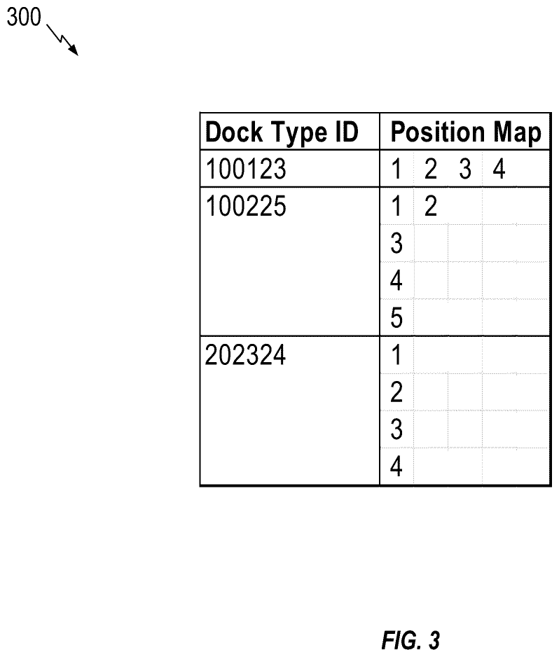

In other example infusion systems, additional or fewer positions may be available for infusion pumps (or other devices), such as based on a type of the infusion pump controller 118. Further, in the depicted example, two of the positions are lateral to the infusion pump controller 118 on a first side, and two on the second side, but other configurations are possible, for example, greater and/or fewer positions on either side. Additionally, in some examples, devices may be positioned vertically, for example, on top and/or below of the infusion pump controller 118. FIG. 3 depicts various configurations of infusion systems.

Each infusion pump 116 may be physically connected to the infusion pump controller 118 through a connector, such as to facilitate a data connection between the infusion pump 116 and the infusion pump controller 118.

### Example Connection Monitoring System

FIG. 1B depicts an example connection monitoring system 100 for detecting, monitoring, and adjusting configurations of modular infusion systems, such as the infusion system 114, coupled to a patient 110.

Infusion system 114 is further configured to transmit and receive data, such as data related to the infusion including infusion parameters, infusion data, operational data, or the like. Infusion system 114 may use network 102 to transmit and receive data. In some aspects, infusion system 114 is configured to transmit data to a hospital information system (HIS) 120 of a medical center of the infusion system 114.

For example, HIS 120 is configured with an electronic medical recordation (EMR) system 122, a pharmacy system 124, a laboratory system 126, a biomedical maintenance system 128, or the like, for the medical center, to facilitate communications such as transmitting and receiving laboratory testing data, prescription data, documentation of a patient's medical history, diagnosis, treatments, laboratory data, medical device status, or the like. HIS 120 may include, or be configured to interface with other hospital systems such as an admissions system, a billing system, a central supply system, unit station systems, or a medical decision support system.

In some aspects, the pharmacy system 124 includes a formulary and/or pharmacy information system. Pharmacy information systems may enable a safer physician medication order process. A pharmacy website (e.g., provided by the server) may provide the physician with a list of available drugs from which the physician may select. The pharmacy website may contain a drug library having the list of available drugs but may also contain and present to the physician the drug names associated with recommended dosages and dose limits that have been established or adopted by the medical center. In such a case where the physician need only select items from the computer screen rather than having to manually type in drug names and drug administration numbers (such as infusion rates, times, etc.) associated with administration of the medication, a more accurate medication process may result.

If a clinical order is for administration of a particular medication regimen, the order will be transmitted to the medical center's pharmacy information system 124. The pharmacy reviews the order, and once the order has been prepared, the order may be transmitted to a nurse station for matching with the appropriate patient. A formulary generally refers to an approved list of drugs for use (e.g., available to order for a patient) within a medical facility. Within a formulary, there may be indication for use information and/or concentrations and drug ranges approved for the medical center. As will be described further, a formulary may be used to define one or more medical device drug libraries, which may then be provided to infusion pumps within HIS 120. Inside the library, there is medication information such as drug names, concentration, diluent volume, strength, minimum or maximum infusion parameters for a drug, and other parameters. The establishment of these parameters, along with parameters for off-formulary orders, via the network 102, is useful for maintaining consistency across the medical center environment and ensuring an order is intelligible and executed according to expectations by other devices within the HIS 120 (e.g., an infusion system 114).

System 100 further includes a connection monitoring system 112. Connection monitoring system 112 is configured to store the data received from the infusion system 114 or the HIS 120 or generated by the connection monitoring system 112 based thereon, in connection data store 104. Connection monitoring system 112 is further configured to analyze the aggregated data in order to, for example, detect coupling of devices of the infusion system 114, determine connection events for the devices, determine recommended contention positions, infusion events, determine maintenance events, or the like, for devices of the infusion system 114.

Although depicted here as separate devices, in some cases, the connection monitoring system 112 may be integral to or form part of infusion system 114 or HIS 120. In some cases, the connection monitoring system 112 may form part of a clinician device, such as a mobile device, a nurse station monitoring device, a computing device, or the like.

The network 102 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

### Example Workflows for Connection and Location Detection

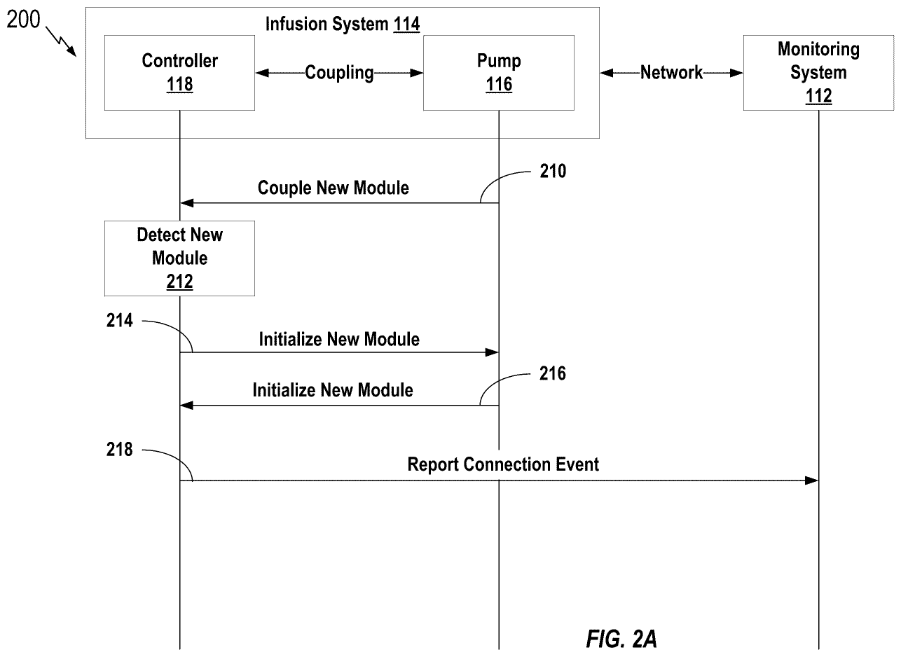

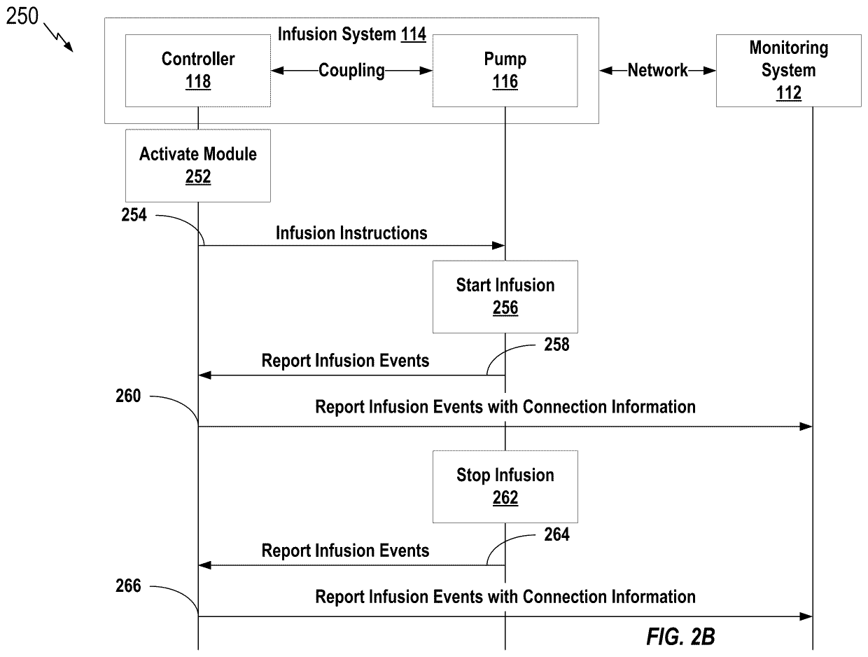

FIG. 2A depicts an example workflow 200 for detecting connections of devices, such as of infusion system 114, and providing connection data to the connection monitoring system 112. In some aspects, the infusion system 114 may use network 102 to transmit and receive data with the connection monitoring system 112, such as described in FIG. 1B.

Initially, at step 210, the pump 116 is coupled to the infusion pump controller 118. Although described as a pump 116, the pump 116 may be any medical device configured for coupling with the infusion system 114.

At step 212, the infusion pump controller 118 detects the coupling of the pump 116. The pump 116 may be detected as coupled to the infusion pump controller 118 through a physical connection, e.g., a wired connection, or sensed through a sensor, such as a pressure sensor. In some aspects, detecting the coupling of the pump 116 with the infusion pump controller 118 may include receiving an identifier of the pump 116 at the infusion pump controller 118. For example, an identifier of the pump 116 may indicate a type of pump 116, a serial number of pump 116, or other information associated with the pump 116.

In some aspects, the pump 116 may be detected as coupled to the infusion pump controller 118 based on an infusion order. For example, an infusion order may include a fluid to be infused by the infusion system 114, as well as one or more operational parameters for the pump 116 to infuse the fluid. In some aspects, an infusion order may further indicate a number and/or type of infusion devices of infusion system 114 to infuse the order. For example, the infusion may indicate the type of infusion pump to infuse the order, a number of infusion pumps to infuse the order, a number of infusion pump controllers to infuse the order, or the like.

At step 214, the infusion pump controller 118 sends information to the pump 116 to initialize the pump 116 for operations. The information may include one or more of: information identifying the pump 116, a type for the pump 116, or software or firmware version information to confirm the pump 116 is compatible with the controller 118.

At step 216, the pump 116 returns information to the infusion pump controller 118 to confirm initialization of the pump 116. The information may include an acknowledgement message or other confirmation. In some implementations, the confirmation may include adjusting the power or the data flowing from the controller 118 to the pump 116.

At step 218, the infusion pump controller 118 sends information to the connection monitoring system 112 regarding the connection event. The information may include an identifier of a dock type of the infusion pump controller 118 and a position of the pump 116. FIG. 3 depicts an example table 300 of various types of docks or infusion pump controllers, and corresponding position maps. For example, for dock type 100123, the position map indicates that up to four devices may be connected in four positions. Pump 116 could be in one of four lateral positions. For dock type 100225, up to five devices may be connected in five positions. Specifically, the first two positions are lateral, and positions three, four, and five are vertical compared to position one. For dock type 202324, up to four devices may be connected in four vertical positions. In some cases, one or more positions may be configured for a type of device, or several types of devices. For example, a first position may be configured for an infusion pump controller, while a second position may be configured for a pump 116. As another example, a first position may be configured for an infusion pump controller, while a second position may be configured for either another infusion pump controller or another pump. Other configurations are possible based on the type of dock, other devices which may be coupled together.

A connection position may be associated with an identifier which indicates a position of the infusion pump 116 relative to the infusion pump controller 118 when connected at that connection position. For example, an identifier of a first connection position may indicate the infusion pump device is infusion pump 116(1) in FIG. 1A.

FIG. 2B depicts an example workflow 250 for detecting infusion events for a connected device, e.g., pump 116. In some aspects, workflow 250 may proceed following workflow 200.

At step 252, the infusion pump controller 118 activates the device, for example, as described with respect to steps 210-216 of workflow 200.

At step 254, the infusion pump controller 118 may send information comprising infusion instructions to the pump 116 for initiating the infusion. An instruction may include a flow rate, a target volume, or other parameter to configure the pump 116 for safe administration of a fluid. At step 256, the pump 116 starts the infusion based on the infusion order information provided by infusion pump controller 118. The pump 116 may start the infusion in response to receiving the order information or based on information included in the order as reviewed and/or confirmed by a clinician.

At step 258, the pump 116 reports infusion events to the infusion pump controller 118, for example, information regarding the operation of the infusion pump 116 to infuse the fluid to the patient. In some aspects, an infusion event may be based on operations of the infusion pump, for example, starting and/or stopping an infusion (e.g., a pause operation), a change to an operational parameter of the infusion, an error of the infusion, an alert or alarm associated with the infusion, a time of operation (e.g., active infusion), or the like.

At step 260, the infusion pump controller 118 sends information regarding the infusion events to the connection monitoring system 112, including information regarding a connection position of the infusion pump 116. A connection position may indicate a position of the pump 116 relative to the infusion pump controller 118, for example, one of the four positions shown in FIG. 1A, or one of the positions of the position map for the dock type shown in FIG. 3.

In some aspects, steps 258-260 may be repeated for each infusion event after starting the infusion at step 256, for example, each time an infusion is pause, or an error occurs. In some aspects, steps 258-260 may be repeated periodically during an infusion, for example, every 5 minutes, hour, 90 minutes, etc. during an infusion.

At step 262, the infusion is stopped. In some cases, the infusion is stopped due to the infusion order being completed, for example, the dose of the fluid to be infused is completed. In some cases, the infusion is stopped due to an error, alarm, or alert of the infusion pump, such as an operational error. In some cases, the infusion is stopped due to clinician or user interaction, for example, where a clinician manually stops the infusion.

At step 264, infusion event information associated with stopping the infusion at step 262 is reported to the infusion pump controller 118.

At step 266, infusion event information and location information are reported to the connection monitoring system 112, including information regarding a connection position of the infusion pump 116.

### Example Workflow for Maintaining Infusion Systems based on Connection Information

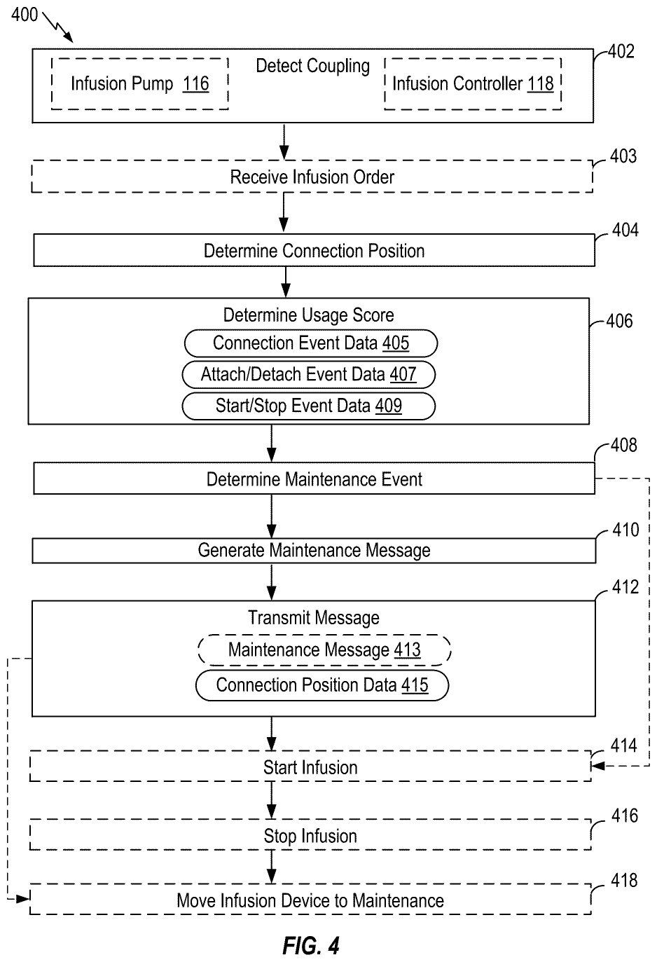

FIG. 4 depicts an example workflow 400 for maintaining infusion systems based on connection information, for example, connection information and connection events generated as described in FIGS. 2A and 2B.

Initially, workflow 400 begins at block 402 with detecting a coupling of two infusion devices, for example, infusion pump 116 with infusion pump controller 118, as described with respect to step 216. In aspects, workflow 400 further includes receiving infusion order information at step 403, for example, information regarding the fluid to be infused, and one or more operational parameters for the infusion.

At block 404, workflow 400 continues with determining a connection position of the first infusion device relative to the second infusion device based on the detected coupling. For example, based on a dock type of the second infusion device, a set of possible connection positions for the first infusion device may be determined. As depicted in FIG. 3, various devices may be associated with various possible connection positions. In some aspects, a second infusion device may have one, two, three, four, five, six, or more connection positions possible. In some aspects, a first infusion device may be configured to connect at one or more of the set of possible connection positions of the second infusion device. Based on the detected coupling, the second infusion device may determine a connection position of the first infusion device. For example, a connection position may be determined based on how the coupling was detected. Detecting the coupling based on a physical connector of the second infusion device and the first infusion device may indicate the connection position of the first infusion device at the physical connector. Detecting the coupling based on a sensor associated with a connection position, may indicate the connection position of the first infusion device at the sensor.

A connection position may be associated with an identifier which indicates a position of the first infusion device relative to the second infusion device when connected at that connection position. For example, an identifier of a first connection position may indicate the first infusion device is infusion pump 116(1) in FIG. 1A.

At block 406, a usage score for the connection position is determined. A usage score indicates usage of the connection position by infusion devices. Because a connector between the two infusion devices may become worn, damaged, or otherwise inoperable due to maintenance, cleaning, and/or repair, a usage score may indicate the usage of the connector. A usage score may be determined based on historical connection data for the infusion devices. For example, a usage score may be based on connection event data 405, attach and detach event data 407, and/or start and stop event data 409.

In some aspects, historical connection data may include connection event data 405 regarding one or more connection events, such as determined at step 218 of FIG. 2A. Historical connection data may be stored in connection data store 104 in FIG. 1B.

In some aspects, historical connection data may include a number of infusion orders of the first infusion device at the connection position. For example, a number of infusion orders of the first infusion device may be based on a number of infusions started while connected to any other infusion device. In some aspects, historical connection data may include a number of infusion orders of the second infusion device at the connection position. For example, a number of infusion orders of the second infusion device may be based on a number of infusions started while connected to any other infusion device.

In some aspects, historical connection data may include a number of attach and detach events at the connection position. An attach and detach event may include a coupling and decoupling of the first infusion device with the second infusion device. For example, an attach event may occur when the first infusion device is coupled with the second infusion device, such as a physical connection. A detach event may occur when the first infusion device is decoupled with the second infusion device, such as a physical disconnection. In some aspects, an attach and detach event may be a coupling and decoupling of the first infusion device with any other infusion device (e.g., other than second infusion device). In some cases, two infusion devices may be attached and detached to facilitate a new configuration of infusion devices, even during an infusion order. Thus, a number of attach and detach events may be greater than a number of infusion orders of the infusion device.

In some aspects, historical connection data may include a start and stop event of the infusion device. A start and stop event may be an operation of the infusion device. A start event may occur when a start operation of the infusion device occurs, while a stop event may occur when a stop operation of the infusion device occurs. In some aspects, a start and stop event may include where an infusion order is paused. For example, an infusion device may be paused and restarted, without decoupling or recoupling of an infusion device. A pause operation may occur where an infusion device pauses operation of an infusion pump. A resume operation may occur where an infusion device resumes operation of an infusion pump. In some cases, a start and stop event may include an error of an infusion device where the infusion operation stops.

In some aspects, historical connection data may include a time period since a previous maintenance event. A previous maintenance event may be associated with a second infusion device, including one or more connection positions of the second infusion device.

In some aspects, historical connection data may include an active time period of the second infusion device. For example, an infusion pump controller may be determined to be active where it is powered on, even when not connected to an infusion pump, or in an infusion operation. As another example, an infusion pump controller may be determined to be active where it is associated with a patient.

In some aspects, historical connection data may include a use time of the first infusion device at the connection position. For example, a use timer may start when an attach event occurs, e.g., a coupling of a first infusion device and a second infusion device. The use timer may stop when a detach even occurs, e.g., a decoupling of the first infusion device and the second infusion device. A use time may be based on the time period between the attach event and the detach event, e.g., using the timer for the attach and detach event. In some examples, a use time may be based on a summation of all timers for all attach and detach events, e.g., a lifetime usage time. In some examples, a use time may be based on a summation of all timers for attach and detach events since a maintenance event, e.g., usage time since last maintenance.

In another example, a use timer may start when a start event occurs, e.g., a start operation of the first infusion device. The use timer may stop when a stop event occurs, e.g., a stop operation of the first infusion device. A use time may be based on the time period between the start event and the stop event, e.g., using the timer for the start and stop event. In some examples, a use timer may be based on a summation of all timers for all start and stop events, e.g., a lifetime usage time. In some examples, a user time may be based on a summation of all timers for start and stop events since a previous maintenance event, e.g., usage time since last maintenance.

In some aspects, a use timer may pause when a pause operation of the infusion device occurs and resume when a resume operation of the infusion device occurs.

In some aspects, a usage score may be based on the infusion order of the infusion devices. For example, where an infusion order indicates an infusion time, the usage score may also include the infusion time. As another example, an infusion order may be associated with a future start and stop event, e.g., based on starting and stopping the infusion order. The usage score may also include the future start and stop event.

In some aspects, a usage score may be determined for each connection position of the set of connection positions at block 406.

At block 408, a maintenance event is determined based on the usage score. A maintenance event may indicate at least one connection position of the set of connection positions of the second infusion device is ready for maintenance, such as a cleaning, repair, or replacement. In some aspects, a maintenance event is determined based on comparing the usage score to an overuse threshold. An overuse threshold may be based on a type of the second infusion device. An overuse threshold may be based on a manufacturer's recommendation, a medical center maintenance schedule, or the like.

In some aspects, a plurality of overuse thresholds may be utilized to generate the maintenance event. A first overuse threshold may indicate a type of maintenance event, for example, a cleaning, replacement, or repair of the connection position. A second overuse threshold may indicate a time period for the maintenance event, for example, a maintenance event before the next infusion event, a maintenance event after the next infusion event, a maintenance event after the next ten infusion events, and the like. In some aspects, an overuse threshold may be based on the historical connection data used to generate the usage score. For example, a time overuse threshold may be associated with use time data, and the maintenance event may be indicated based on the use time of the infusion device. In some aspects, a time period for the maintenance event may be based on the infusion order, e.g., where the infusion order is included in the usage score, an overuse threshold indicates the maintenance event after the infusion order is completed. As another example, inclusion of the infusion order in the usage score may indicate satisfaction of an overuse threshold, and the maintenance event is indicated as before the infusion order starts.

In some aspects, each usage score for each connection position may be compared to the overuse threshold. Where at least one usage score satisfies the overuse threshold, a maintenance event may be determined.

In some aspects, the plurality of overuse thresholds may include one or more different thresholds for different connection positions. For example, a first connection position may be associated with a first overuse threshold, while a second connection position may be associated with a second overuse threshold. In some cases, different connection positions may be associated with different types of devices, and the overuse threshold for each connection position may depend on a type of device for the connection position. In some cases, different connection positions may be associate with different thresholds based on other factors, such as type of the connection position, cleaning or maintenance of the connection position, or other characteristic of the connection position.

In some aspects, a respective usage score for each connection position may be compared to a respective overuse threshold for the respective connection position of the set of connection positions. Where at least one usage score satisfies the respective overuse threshold, a maintenance event may be determined.

In some aspects, the usage score of any connection position of the set of connection positions does not satisfy the overuse threshold and workflow 400 may proceed to block 414 with starting the infusion.

At block 410, a maintenance message 413 is generated based on the determination of a maintenance event. A maintenance message may include an identifier indicating the second infusion device. A maintenance message may further indicate an identifier of the at least one connection position with the usage score satisfying the overuse threshold. Where more than one connection position has a usage score satisfying the overuse threshold, the maintenance message may include an identifier of each connection position with a usage score satisfy the overuse threshold.

At block 412, the maintenance message 413 and connection position data 415 is transmitted to the connection monitoring system 112. In some aspects, the maintenance message 413 may also be transmitted to the HIS 120, for example, to the biomed maintenance system 128 to schedule maintenance for the infusion device. The maintenance message 413 may indicate the one or more connection positions ready for maintenance, e.g., based on usage score. In some aspects, a biomed maintenance system 128, or other system part of HIS 120, may coordinate the maintenance of the infusion device. For example, where the overuse threshold indicates a maintenance event after the next infusion order, the biomed maintenance system 128 may schedule the maintenance based on the infusion time. As another example, where the maintenance event indicates a repair, the biomedical maintenance system 128 may determine, based on the maintenance message 413, to send the infusion device for an off-site maintenance, such as at a manufacturer's facility, or a servicer's facility.

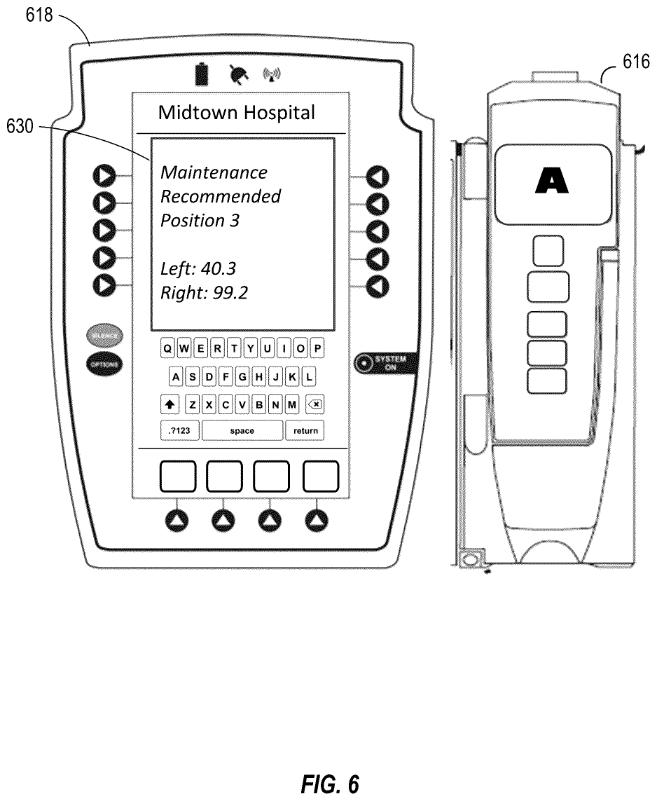

In some aspects, transmission of the maintenance message 413 and the connection position data 415 includes outputting at the second infusion device, a maintenance alert. The maintenance alert may include the maintenance message 413 to alert a clinician the infusion device is ready for maintenance. For example, the maintenance alert may be displayed on a user interface, the maintenance alert may be outputted as an audio output, e.g., audible alert, the maintenance alert may be outputted as a visual alert, e.g., a light or other visual signal, or the like. FIG. 6 depicts an example infusion pump controller 618 connected to an infusion pump 616, with a user interface 630 displaying a maintenance message and usage scores for two positions (e.g., left, and right) of the infusion pump controller 618. In such cases, workflow 400 may proceed to block 418, where the infusion device is moved to a maintenance facility, such as associated with biomed maintenance system 128.

In some cases where the maintenance message 413 indicates a future maintenance event, such as after the infusion order completes, the maintenance alert may alert the clinician to the future infusion event. Then, workflow 400 may proceed to block 414 with starting the infusion order. At block 416, the infusion is stopped. At block 418, the infusion device is moved to the maintenance facility for maintenance.

Note that workflow 400 is just one example, and other flows including fewer, additional, or alternative steps, consistent with this disclosure, are possible.

### Example Workflow for Recommending Connection Positions

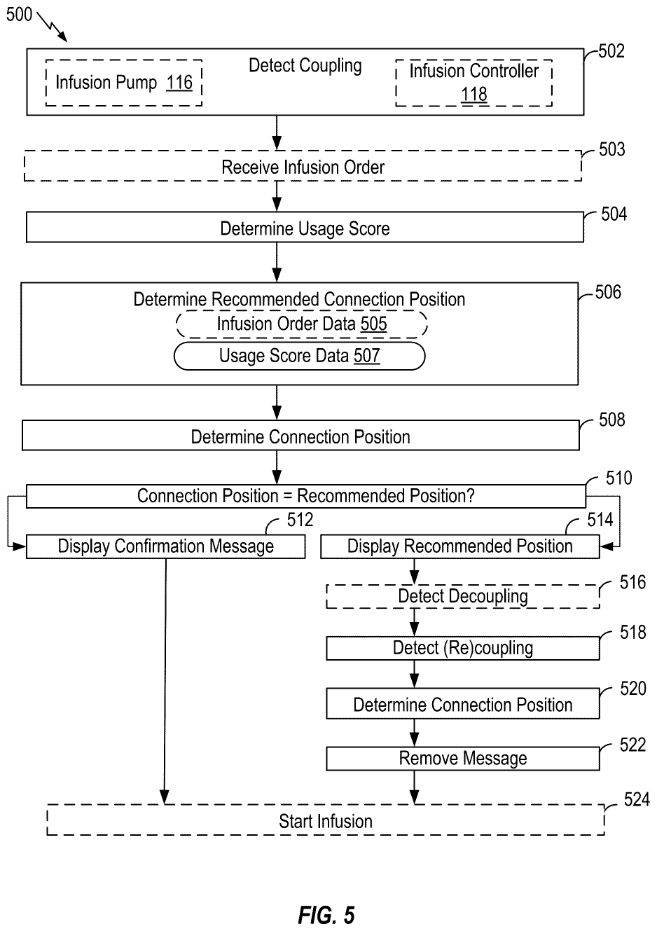

FIG. 5 depicts an example workflow 500 for maintaining infusion systems based on connection information, for example, connection information and connection events generated as described in FIGS. 2A and 2B. In some cases, connection positions for a first infusion device may be recommended when connecting to a second infusion device to maintain the infusion device, e.g., reduce maintenance events due to overuse of connection positions. In some cases, connection positions for a first infusion device may be recommended to establish efficient configurations, for example, where multiple infusion devices may be connected.

Initially, workflow 500 begins at block 502 with detecting a coupling of two infusion devices, for example, infusion pump 116 with infusion pump controller 118, as described with respect to step 216 and block 402. In aspects, workflow 500 further includes receiving infusion order information at step 503, for example, information regarding the fluid to be infused, and one or more operational parameters for the infusion. In some aspects, infusion order information is received prior to detecting the coupling of the two infusion devices, and the coupling is detected based on the infusion order. For example, the infusion order may indicate a type and/or number of infusion devices for the infusion order.

Workflow 500 continues at block 504 with determining a usage score for each connection position of the connection positions of the second infusion device. For example, based on a dock type of the second infusion device, a set of possible connection positions for the first infusion device may be determined. As depicted in FIG. 3, various devices may be associated with various possible connection positions. In some aspects, a second infusion device may have one, two, three, four, five, six, or more connection positions possible. In some aspects, a first infusion device may be configured to connection at one or more of the set of possible connection positions of the second infusion device.

A usage score for each connection position of the connection positions of the second infusion device may be determined as described with respect to block 406 of FIG. 4. For example, a usage score may be based on historical connection data for the second infusion device.

At block 506, workflow 500 continues with determining a recommended connection position. A recommended connection position may be one connection position of the set of connection positions of the second infusion device. For example, a recommended connection position may be a third position for the first infusion device, e.g., pump 116(3) at position three in FIG. 1A.

A recommended connection position may be determined based on a usage score of each connection position, for example, stored as usage score data 507 in connection data store 104 in FIG. 1B. For example, a recommended connection position may be determined by comparing the usage scores of each connection position to a usage threshold. A usage threshold may indicate a connection position may be used before a maintenance event. A usage threshold may be based on an average usage of the set of connection positions of the second infusion device. A usage threshold may be based on an average usage of connection positions of infusion devices of the same type as the second infusion device.

A usage score for a connection position may satisfy the usage threshold where the usage score is below the usage threshold, indicating the connection position may be used.

In some aspects, a plurality of usage thresholds may include one or more different thresholds for different connection positions. For example, a first connection position may be associated with a first usage threshold, while a second connection position may be associated with a second usage threshold. In some cases, different connection positions may be associated with different types of devices, and the usage threshold for each connection position may depend on a type of device for the connection position. In some cases, different connection positions may be associated with different thresholds based on other factors, such as type of the connection position, cleaning or maintenance of the connection position, or other characteristic of the connection position.

In some aspects, a respective usage score for each connection position may be compared to a respective usage threshold for the respective connection position of the set of connection positions. Where a usage score satisfies the respective usage threshold for that connection position, the connection position may be used.

In some aspects, multiple connection positions may be recommended, for example, a first position and a third position for the first infusion device. Recommended connection positions may be recommended in a priority or preference order. For example, a third position may be recommended first, while a first position may be recommended second. The priority or preference order may be based on the usage score of each recommended position, for example, for the first and third connection positions.

Where the usage scores of two or more connection positions satisfy the usage threshold, both connection positions may be recommended. A priority level for each recommended connection position may be assigned based on the usage scores of each connection position. A priority level may be assigned based on comparing the usage score of each connection position, for example, a lower usage score may be assigned a higher priority level.

A priority level may be based on a difference between the usage threshold and the usage score for a connection position. For example, a higher priority level may be assigned to a connection position with a usage score with a greater difference between the connection position's usage score and the usage threshold, compared to a second connection position with a lesser difference between the connection position's usage score and the usage threshold.

A priority level may be based on a recent maintenance event of the connection position. For example, a connection position with a more recent maintenance event may be assigned a higher priority level compared to a connection position with an older maintenance event.

In some aspects, a recommended connection position may be based on the infusion order data 505. For example, where an infusion order indicates an infusion time, the usage score may also include the infusion time. As another example, an infusion order may be associated with a future start and stop event, e.g., based on starting and stopping the infusion order. The usage score may also include the future start and stop event.

In some aspects, the infusion order data 505 may indicate one or more infusion orders at the second infusion device, for example, where multiple devices may be coupled to the second infusion device. A recommended connection position may be based on the one or more additional infusion orders. For example, a first recommended connection position may be recommended for the first infusion order and a second recommended connection position may be recommended for the second infusion order.

In some aspects, the recommended connection position for a first infusion device may be based on comparing an infusion time of the first infusion order and the second infusion order. For example, the infusion order with the longer infusion time may be recommended to a higher priority connection position. An infusion order with a longer infusion time may be recommended to an inner position (e.g., position two or position three in FIG. 1A) or closer to the second infusion device to reduce reconfiguration of the infusion system.

In some aspects, the recommended connection position for the first infusion device may be based on a type of fluid of the first infusion order and the second infusion order. In some aspects, the recommended connection position for the first infusion device may be based on a type of device of the first infusion order, e.g., a type of the first infusion device.

Workflow 500 then continues to block 508 with determining a connection position of the first infusion device, such as described at block 404 of workflow 400. For example, based on how the coupling was detected, the connection position may be determined. In some aspects, the coupling was detected based on a wireless coupling between the first and second infusion devices and the connection position of the first infusion device is determined as not connected to the second infusion device.

At block 510, the connection position is compared to the recommended connection position. If the connection position is the same as the recommended connection position, for example, both indicated as position one, then the workflow proceeds to block 512 with outputting a confirmation message confirming the connection of the first infusion device and the second infusion device at the recommended connection position on the second infusion device. For example, a confirmation message may be displayed on a user interface of the second infusion device. Workflow 500 may then proceed to block 524 with starting the infusion.

If, at block 510, the connection position is not the same as the recommended connection position, for example, where the connection position is a first position and the recommended connection position is a third position, workflow 500 proceeds to block 514.

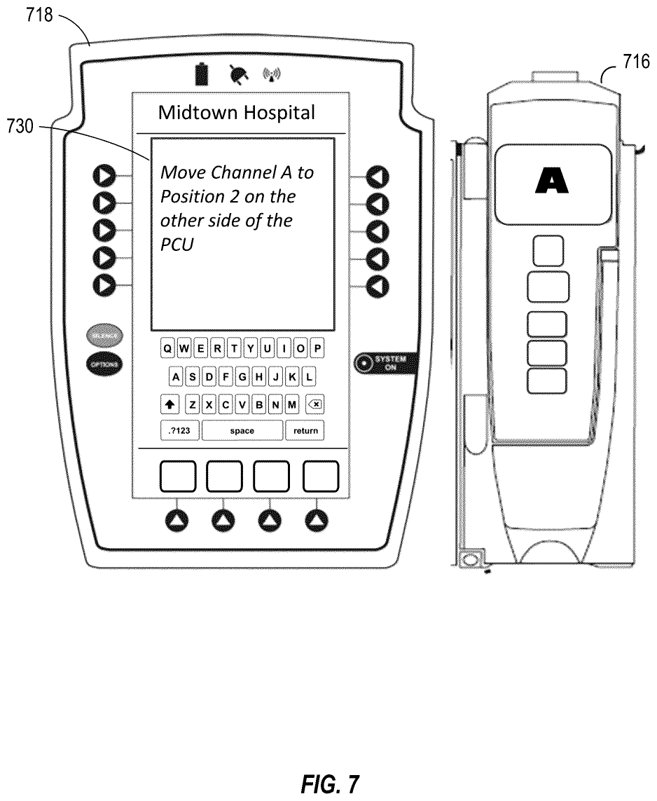

At block 514, a recommendation message may be outputted on the second infusion device. The recommendation message may indicate the recommended position. For example, a recommendation message may be displayed on a user interface of the second infusion device. FIG. 7 depicts an example infusion pump controller 718 connected to an infusion pump 716, with a user interface 730 displaying a recommendation message, in this case, to move the infusion pump 716 at channel A to position 2 on the other side of the infusion pump controller 718.

Where the first infusion device is determined as not connected to the second infusion device, the recommendation message may indicate to physically connect the first infusion device at the recommended connection position. Workflow 500 may then continue to block 518 with detecting a coupling of the first infusion device, for example, based on a physical connection through a connector or a sensor.

Where the connection position of the first infusion device is not the same as the recommended connection position, the second infusion device may detect a decoupling of the first infusion device at block 516. For example, a decoupling of the first infusion device may be detected based on disconnection of the first infusion device.

The second infusion device detects a recoupling of the first infusion device at block 518. Based on the detected recoupling of the first infusion device, the connection position of the first infusion device may be determined at block 520, similarly to at block 508. Where the connection position is the same as the recommended connection position, workflow 500 proceeds with removing the recommendation message at block 522. In some aspects, a confirmation message, such as described at block 512 may be outputted.

If the connection position determined at block 520 is not the same as the recommended connection position, the workflow 500 may repeat steps 516-520 until it is determined as the same.

If, at block 506 multiple recommended connection positions are determined, workflow 500 may proceed to block 512 where the connection position is one of the multiple recommended connection positions. In some aspects, if, at block 506 multiple recommended connection positions are determined, workflow 500 may proceed to block 512 where the connection position is the highest priority recommended connection positions, workflow 500 may proceed to block 512.

In some aspects, where multiple recommended connection positions are determined and the connection position is one of the multiple recommended connection positions, but not the highest priority, then workflow 500 may proceed to block 514 with outputting the recommended connection position. If at block 514, no decoupling is detected, for example, within a time period, workflow 500 may proceed to block 512.

If, at block 506 at block 506 multiple recommended connection positions are determined, workflow 500 may proceed to block 514 where the connection position is none of the multiple recommended connection positions.

At block 524, the infusion may start.

Note that workflow 500 is just one example, and other flows including fewer, additional, or alternative steps, consistent with this disclosure, are possible.

### Example Infusion System

FIG. 8 depicts an example infusion system 800, which may implement methods described herein for device management. The infusion system 800 may be coupled to a patient 850 to administer a substance to the patient 850 through infusion. The infusion system 800 may include one or more pumps, for example, pump 802, pump 804, pump 806, and pump 808. Although a large volume pump is illustrated, other types of pumps may be implemented, such as a peristaltic pump, a small volume pump, a syringe pump, an anesthesia delivery pump, or a patient-controlled analgesic. A pump may be an infusion device configured to deliver a substance, (e.g., fluid, nutrients, drug, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 802, 804, 806, or 808 may comprise a standard infusion pumping unit, patient-controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+ ml/hr).

Each of the pumps 802, 804, 806, or 808 may be fluidly connected with an upstream fluid line 812, fluid line 814, fluid line 816, and fluid line 818, respectively. Further, each of pump 802, pump 804, pump 806, and pump 808, may be fluidly connected with a downstream fluid line 822, fluid line 824, fluid line 826, and fluid line 828, respectively. The fluid lines may be any type of fluid conduit, such as tubing, through which fluid can flow.

Each of fluid supply 832, fluid supply 834, fluid supply 836, and fluid supply 838, may be a reservoir, for example, as bottles shown, inverted, and suspended above the pumps. Fluid supplies may also take the form of bags, syringes, or other types of containers. The system 800 may be mounted on a roller stand or intravenous pole 840.

Infusion pump system 800 may further comprise check valves, drip chambers, valved ports, connectors, and other devices configured to administer a substance.

Each of the pumps 802, 804, 806, or 808 of infusion pump system 800 may be coupled to an infusion pump controller 860. The infusion pump controller 860 may be a patient care device (PCD) or patient care unit (PCU). The infusion pump controller 860 is configured to control operations of the pumps 802, 804, 806, or 808, for example, to control infusions to patient 850. The infusion pump controller 860 may be further configured to the control of fluid delivery to the patient and the monitoring of the fluid path for occlusion or air-in-line obstructions.

The infusion pump controller 860 may further include a graphical user interface (GUI), for example, an information display such as a liquid crystal display, and/or a touch-screen. The user interface of the infusion pump controller 860 may be used during set up and/or operating procedures to facilitate control of the infusion system 800. For example, entry, editing, and/or display of operational parameters of the pumps 802, 804, 806, or 808. The infusion pump controller 860 may further include one or more softkeys and/or hardkeys to facilitate data entry and commands. In some aspects, a user interface may display the operational parameters of the pumps 802, 804, 806, or 808, for example, the infusion rate at which the pump is operating. The user interface may additionally and/or alternatively, be used to display informational, advisory, alarm, or malfunction messages associated with the pumps 802, 804, 806, or 808.

The infusion pump controller 860 may further be configured to provide power to the infusion system 800 and interface between aspects of the infusion system 800 and external devices and/or networks, for example, patient monitoring networks, instructional systems (e.g., an electronic medical record system), pharmacy systems, and/or nurse call systems, or as an interface to external equipment such as barcode readers to provide a means of inputting drug and/or patient information from medication or patient records. In some aspects, the infusion pump controller 860 is configured to provide physical attachment of the infusion system 800 to structures, such as intravenous pole 840 and/or bedrails, and the like.

### Example Syringe Pump

FIG. 9 depicts an example syringe pump 902. Syringe pump 902 includes a graphical user interface (GUI) 924 for displaying operational information, including the selected syringe type and the configured infusion (e.g., in terms of rate of infusion, volume infused, length of time of infusion, etc.). A user may operate syringe pump 902 through one or more buttons 922 in this example. Note that other examples may include other arrangements, such as touch-screen interfaces, or interfaces operable by remote device, such as by an application on a computing device.

Syringe 901 is shown next to syringe pump 902, rather than loaded in the pump, for clarity of illustration. Syringe pump 902 includes cradle 904 in which a barrel 903 of syringe 901 rests when mounted in the syringe pump 902. Sensor 928 detects volume markings along a barrel of syringe 901. Cradle 904 has a clamp 906 to securely hold the barrel 903 in a fixed position in cradle 904 to resist axial and lateral movement. Clamp 906 may pivot between an open position to permit loading or removal of syringe 901 and a closed position over cradle 904. Barrel clamp 906 may measure an outside diameter of the barrel 903 of syringe 901. Barrel flange 905 of syringe 901 resides in a barrel flange groove 908 in syringe pump 902 to immobilize barrel 903 from axial movement during movement of plunger 907 within barrel 903 of syringe 901. Barrel flange groove may measure an outside diameter and a thickness of the barrel flange 905 of syringe 901.

Plunger 907 can include push-button 909 having an inner side 911 and being interconnected with stopper 913 of plunger 907 by piston 915. When mounted in syringe pump 902, push-button 909 can be held by drive head 910 with a plunger retainer comprising a pair of pivotally mounted claws, first retainer claw 912 and second retainer claw 914, shown in the closed position in FIG. 9. The retainer claws 912 and 914 can curve inwardly toward each other to grasp push-button 909 mounted in syringe pump 902.

A rotation knob 916 can be used to control the positions of the first and second retainer claws 912 and 914 to allow removal and insertion of the push-button 909 and to release the split-nut from the driveshaft to permit axial positioning of the drive head 910. Syringes can be provided for use with a syringe pump with different quantities of fluid, and the plunger can be located at different positions in relation to the barrel.

The drive head 910 can allow manual adjustment to accommodate syringes with different beginning plunger positions. A syringe inserted in the cradle 904 can align with the drive head 910 within a particular axial range. The points where the axial center lines of the syringe intersect the driver can change according to the size of the syringe but only in one direction along the drive head 910. A guide device 918 can extend from the drive head 910 to a point within a body of the syringe pump 902.

Syringe pump 902 can include a control panel 920 providing multiple buttons 922 for control of the syringe pump 902 as well as GUI 924 used to present pump-specific information to the operator. The buttons 922 can allow the operator to program the syringe pump 902 for the flow rate, the volume to be infused, and other pump parameters. GUI 924 can present the programmed flow rate, the amount of fluid remaining to be infused, as well as alarms and other information.

The drive head 910 can include a contact plate 926 that has a pushing surface that contacts the outer side 917 of the push-button 909 as the drive head 910 moves forward toward the barrel 903, pushing the plunger 907 into the barrel 903 of the syringe to expel the syringe contents through a fluid administration set tubing 919 to a patient. A drive mechanism 954 is configured to drive the drive head 910 forward. The drive mechanism 954 may include, a drivetrain, for example, gears, axles, shafts, chains, hydraulics, and/or any other components for translating rotational motion of a motor into linear motion of the drive head 910. The drivetrain may include linear actuating components, such as a linear stepper motor, a brushed DC electric motor, a brushless DC electric motor, a servo motor, an AC motor, or any other type of motor.

When the contact plate 926 exerts force against the push-button 909, the force may be detected by force sensor 929 and transmitted to a processor for monitoring. In the case of friction between the stopper 913 and the barrel 903, the force exerted can increase and can be detected by force sensor 929.

Syringe pump 902 is configured with known syringe types containing information such as syringe inner diameter and stroke of the stopper 913. Syringe pump 902 determines the position of stopper 913 based on the movement of drive head 910 based on the syringe type and stored characteristics of the syringe type. Then, syringe pump 902 calculates the volume infused, time elapsed, volume remaining, and time remaining. As the drive head 910 continues to move, a flow rate is determined based on the characteristics of the syringe and the velocity of the drive head 910.

One or more sensors, such as sensor 928, barrel clamp 906, groove 908, claws 912 and 914, and force sensor 929, may measure various characteristics of syringe 901, to obtain a measured value for the characteristic, such as barrel length, barrel outside diameter, flange shape, flange size, plunger shape, plunger size (e.g., diameter), plunger tab thickness, volume per plunger distance moved, driver head height, etc. These sensors may include one or more of an optical sensor, a light source, a pressure sensor, a position sensor (e.g., magnetic linear position sensor indicating the plunger head), force sensor, and the like.

### Example Method for Recommending Connection Positions

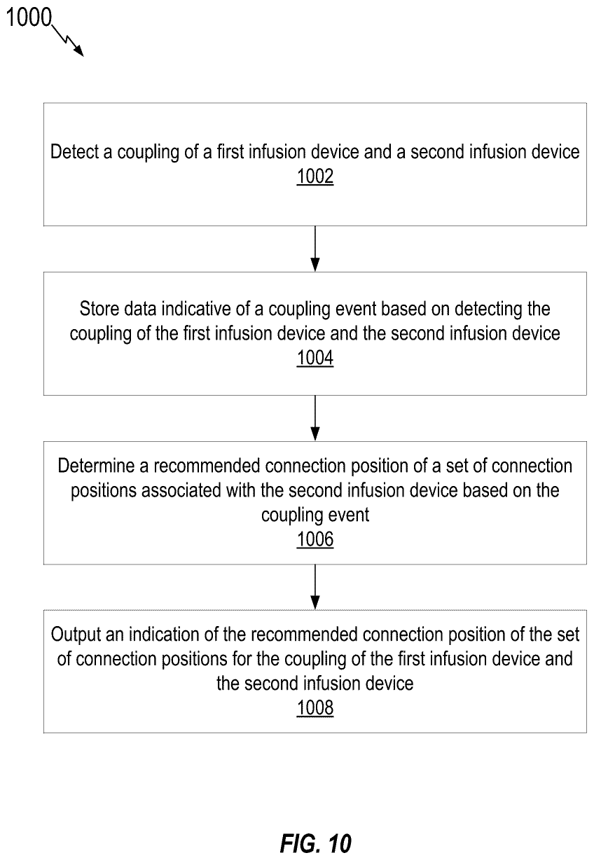

FIG. 10 shows an example method 1000 for device configuration management, including for an infusion system, such as infusion system 800 in FIG. 8 or syringe system 900 in FIG. 9.

Method 1000 begins at 1002 with detecting a coupling of a first infusion device and a second infusion device.

Method 1000 proceeds with 1004 storing data indicative of a coupling event based on detecting the coupling of the first infusion device and the second infusion device.

Method 1000 then proceeds with 1006 with determining a recommended connection position of a set of connection positions associated with the second infusion device based on the coupling event.

In some aspects, determining the recommended connection position of the set of connection positions based on the coupling event comprises: generating a usage score based on historical connection data for each connection position in the set of connection positions; comparing the usage score for each connection position in the set of connection positions to an usage threshold for the set of connection positions; and determining the recommended connection position based on the usage score of one or more connection positions in the set of connection positions satisfying the usage threshold.

In some aspects, for each connection position in the set of connection positions, the historical connection data comprises a number of start and stop operations of the second infusion device while coupled at the connection position.

In some aspects, the historical connection data comprises a number of attach and detach events of the second infusion device.

In some aspects, the historical connection data comprises a time period since a previous maintenance event.

In some aspects, the historical connection data comprises an active time period of the second infusion device.

In some aspects, the recommended connection position of the set of connection positions is further based on one or more operational parameters of the first infusion device. In some aspects, the one or more operational parameters comprise a flow rate, a volume, a velocity, or an infusion time for the first infusion device.

In some aspects, the method further comprises detecting a second coupling of the second infusion device and one or more additional infusion devices, wherein the recommended connection position is further based on the one or more additional infusion devices.

In some aspects, the recommended connection position is further based on one or more operational parameters of the one or more additional infusion devices.

In some aspects, the coupling of the first infusion device and the second infusion device comprises a physical connection at a first connection position of the set of connection positions.

In some aspects, the method further comprises outputting a connection confirmation indication.

Method 1000 proceeds with 1008 with outputting an indication of the recommended connection position of the set of connection positions for the coupling of the first infusion device and the second infusion device.

In some aspects, the recommended connection position is the same as the first connection position of the set of connection positions; and the method further comprises: outputting a connection confirmation indication confirming the coupling of the first infusion device and the second infusion device.

In some aspects, the method further comprises in response to determining the connection position of the first infusion device to the second infusion device after the recoupling is the same as the recommended connection position, initiating a start operation of the first infusion device.

In some aspects, the recommended connection position is different from the first connection position of the set of connection positions, and the method further comprises: outputting an indicator indicating to couple the first infusion device and the second infusion device at the recommended connection position. In some aspects, the method further comprises: detecting a decoupling of the first infusion device from the second infusion device; detecting a recoupling of the first infusion device to the second infusion device; determining a connection position of the first infusion device to the second infusion device based on the recoupling; comparing the connection position of the first infusion device to the second infusion device after the recoupling to the recommended connection position; and in response to determining the connection position of the first infusion device to the second infusion device after the recoupling is the same as the recommended connection position, removing the indicator indicating to couple the first infusion device and the second infusion device at the recommended connection position.

In some aspects, the first infusion device comprises an infusion pump. In some aspects, the second infusion device comprises an infusion docking station. In some aspects, the second infusion device comprises an infusion controller.

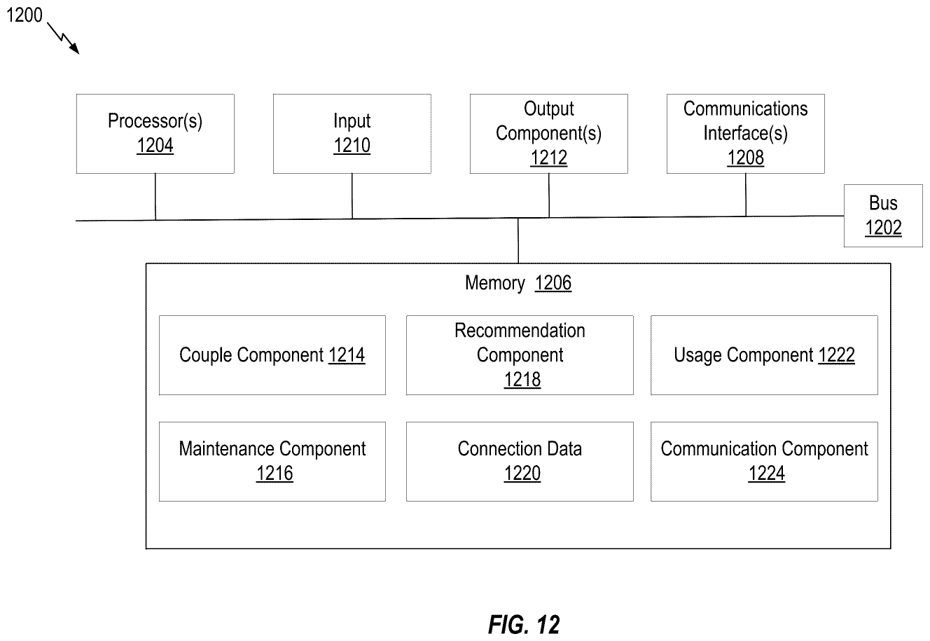

In one aspect, method 1000, or any aspect related to it, may be performed by an apparatus, such as computing device 1200 of FIG. 12, which includes various components operable, configured, or adapted to perform the method 1000. Computing device 1200 is described below in further detail.

Note that FIG. 10 is just one example of a method, and other methods including fewer, additional, or alternative steps are possible consistent with this disclosure.

### Example Method for Maintenance Events

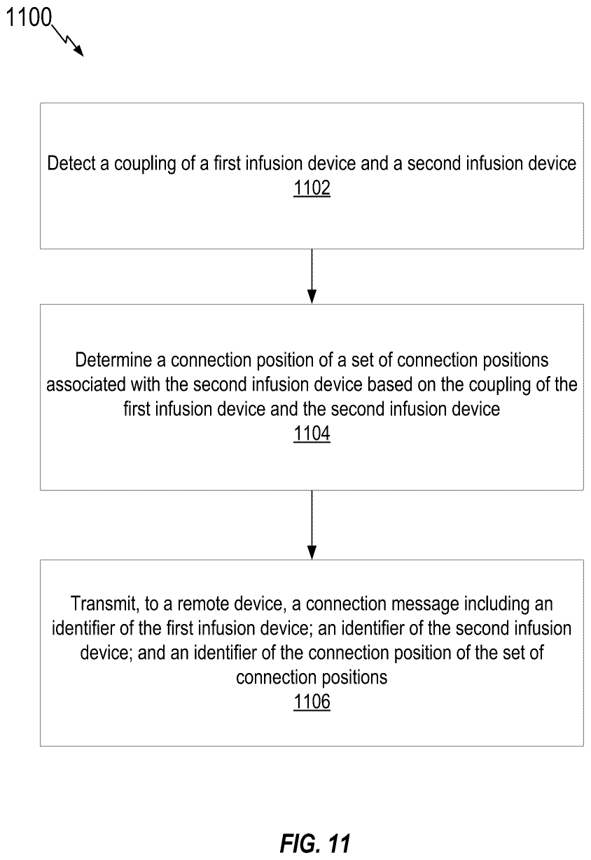

FIG. 11 shows an example method 1100 for device configuration managing including for an infusion system, such as infusion system 800 in FIG. 8 or syringe system 900 in FIG. 9.

Method 1100 begins at 1102 with detecting a coupling of a first infusion device and a second infusion device.

Method 1100 proceeds with 1104 with determining a connection position of a set of connection positions associated with the second infusion device based on the coupling of the first infusion device and the second infusion device.

Method 1100 then proceeds with 1106 with transmitting, to a remote device, a connection message including an identifier of the first infusion device; an identifier of the second infusion device; and an identifier of the connection position of the set of connection positions.

In some aspects, the identifier of the connection position of the set of connection positions indicates a position of the coupling of the first infusion device relative to the second infusion device.

In some aspects, the connection message further includes a usage score of each connection position in the set of connection positions.

In some aspects, the method further comprises generating a usage score based on historical connection data for each connection position in the set of connection positions.

In some aspects, the historical connection data comprises an attach and detach event of the second infusion device.

In some aspects, the method further comprises starting a timer based on the coupling of the first infusion device and the second infusion device; detecting a decoupling of the first infusion device and the second infusion device; stopping the timer based on the decoupling of the first infusion device and the second infusion device; and determining the attach and detect event based on the coupling of the first infusion device and the second infusion device and the decoupling of the first infusion device and the second infusion device, and a time period based on the timer associated with the attach and detach event of the second infusion device.

In some aspects, the historical connection data comprises a start and stop event of the second infusion device. In some aspects, the method further comprises initiating a start operation of the first infusion device; starting a timer based on the start operation of the first infusion device and the second infusion device; initiating a stop operation of the first infusion device; stopping the timer based on the stop operation of the first infusion device and the second infusion device; and determining the start and stop event of the second infusion device based on the start operation and the stop operation, and a time period based on the timer associated with the start and stop event.

In some aspects, the method further comprises initiating a pause operation of the first infusion device; pausing the timer based on the pause operation of the first infusion device; initialing a resume operation of the first infusion device; and resuming the timer based on the resume operation of the first infusion device.

In some aspects, the historical connection data comprises a time period since a previous maintenance event. In some aspects, the historical connection data comprises an active time period of the second infusion device.

In some aspects, the method further comprises generating a maintenance message based on the usage score of one or more connection positions in the set of connection positions. In some aspects, generating the maintenance message based on the usage score of one or more connection positions in the set of connection positions, comprises: comparing the usage score for each connection position in the set of connection position to an overuse threshold for the set of connection positions; and determining a maintenance event for at least one connection position of the set of connection positions based on the usage score of one or more connection positions in the set of connection positions satisfying the overuse threshold, wherein the maintenance message indicates an identifier of the at least one connection position of the set of connection positions.

In some aspects, the coupling of the first infusion device and the second infusion device comprises a physical connection at a first connection position of the set of connection positions.

In some aspects, the first infusion device comprises an infusion pump. In some aspects, the second infusion device comprises an infusion docking station. In some aspects, the second infusion device comprises an infusion controller.

In one aspect, method 1100, or any aspect related to it, may be performed by an apparatus, such as computing device 1200 of FIG. 12, which includes various components operable, configured, or adapted to perform the method 1100. Computing device 1200 is described below in further detail.

Note that FIG. 11 is just one example of a method, and other methods including fewer, additional, or alternative steps are possible consistent with this disclosure.

### Example Computing Device

FIG. 12 depicts an example computing device 1200, such as a medical device, which implements various features and processes described herein, such as infusion system 800 in FIG. 8 and/or syringe pump 902. For example, the computing device 1200 may perform one or more steps of any of workflow 200, 250, 400, or 500 or method 1000 or 1100. The computing device 1200 may include one or more processors 1204, one or more memories 1206, one or more input components 1210, one or more output components 1212, and one or more communication interfaces 1208. Each of these components may be coupled by a bus 1202.

Computing device 1200 may perform these processes based on one or more processors 1204 executing software instructions stored by a computer-readable medium, such as one or more memories 1206. In certain embodiments, one or more processors 1204 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 1208. When executed, software instructions stored in one or more memories may cause one or more processors 1204 to perform one or more processes described herein.

A memory 1206 may include data storage or one or more data structures (e.g., a database, etc.). Computing device 1200 may be capable of receiving information from, storing information in, communicating information to, or searching information stored in the data storage or one or more data structures in one or more memories 1206.

A memory 1206 may include random access memory (RAM), read only memory (ROM), and/or other types of dynamic or static storage devices (e.g., flash memory, magnetic memory, optical memory, etc.), that stores information and/or instructions for use by one or more processors 1204. For example, a memory 1206 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, one or more memories 1206 may include couple component 1214 configured for detecting a coupling of a first infusion device and a second infusion device; detecting a decoupling of the first infusion device from the second infusion device; and detecting a recoupling of the first infusion device to the second infusion device.

In some aspects, one or more memories 1206 may include maintenance component 1216 configured for generating a maintenance message based on the usage score of one or more connection positions in the set of connection positions.

In some aspects, one or more memories 1206 may include recommendation component 1218 configured for determining a recommended connection position of a set of connection positions associated with the second infusion device based on the coupling event.

In some aspects, one or more memories 1206 may include usage component 1222 configured for generating a usage score based on historical connection data for each connection position in the set of connection positions; and determining a connection position of a set of connection positions associated with the second infusion device based on the coupling of the first infusion device and the second infusion device.

In some aspects, one or more memories 1206 may include communication component 1224 configured for storing data indicative of a coupling event based on detecting the coupling of the first infusion device and the second infusion device; outputting an indication of the recommended connection position of the set of connection positions for the coupling of the first infusion device and the second infusion device

In some aspects, one or more memories 1206 may include connection data 1220 used by recommendation component 1218, usage component 1222, or maintenance component 1216.

One or more processors 1204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 1210 may include a component that permits computing device 1200 to receive information, such as via user input (e.g., a touch-screen display, a keyboard, a keypad, a mouse, a button, a switch, a microphone, etc.). Further, one or more input components 1210 may include a sensor for sensing information (e.g., a global positioning system (GPS) component, an accelerometer, a gyroscope, an actuator, etc.).

One or more output components 1212 may include a component that provides output information from computing device 1200 (e.g., a display, a speaker, one or more light-emitting diodes (LEDs), etc.).

Communication interface 1208 may include a transceiver-like component (e.g., a transceiver, a separate receiver and transmitter, etc.) that enables computing device 1200 to communicate with other devices, such as via a wired connection, a wireless connection, or a combination of wired and wireless connections. Communication interface 1208 may permit computing device 1200 to receive information from another device and/or provide information to another device. For example, communication interface 1208 may include an Ethernet interface, an optical interface, a coaxial interface, an infrared interface, a radio frequency (RF) interface, a universal serial bus (USB) interface, a Wi-Fi interface, a cellular network interface, and/or the like.

### Example Clauses

Implementation examples are described in the following numbered clauses:

Clause 1: A method, comprising: detecting a coupling of a first infusion device and a second infusion device; storing data indicative of a coupling event based on detecting the coupling of the first infusion device and the second infusion device; determining a recommended connection position of a set of connection positions associated with the second infusion device based on the coupling event; and outputting an indication of the recommended connection position of the set of connection positions for the coupling of the first infusion device and the second infusion device.

Clause 2: The method of clause 1, wherein determining the recommended connection position of the set of connection positions based on the coupling event comprises: generating a usage score based on historical connection data for each connection position in the set of connection positions; comparing the usage score for each connection position in the set of connection positions to an usage threshold for the set of connection positions; and determining the recommended connection position based on the usage score of one or more connection positions in the set of connection positions satisfying the usage threshold.

Clause 3: The method of clause 2, wherein, for each connection position in the set of connection positions, the historical connection data comprises a number of start and stop operations of the second infusion device while coupled at the connection position.

Clause 4: The method of any one of clauses 2-3, wherein the historical connection data comprises a number of attach and detach events of the second infusion device.

Clause 5: The method of any one of clauses 2-4, wherein the historical connection data comprises a time period since a previous maintenance event.

Clause 6: The method of any one of clauses 2-5, wherein the historical connection data comprises an active time period of the second infusion device.

Clause 7: The method any one of clauses 1-6, wherein the recommended connection position of the set of connection positions is further based on one or more operational parameters of the first infusion device.

Clause 8: The method of clause 7, wherein the one or more operational parameters comprise a flow rate, a volume, a velocity, or an infusion time for the first infusion device.

Clause 9: The method any one of clauses 1-8, further comprising detecting a second coupling of the second infusion device and one or more additional infusion devices, wherein the recommended connection position is further based on the one or more additional infusion devices.

Clause 10: The method of clause 9, wherein the recommended connection position is further based on one or more operational parameters of the one or more additional infusion devices.

Clause 11: The method any one of clauses 1-10, wherein the coupling of the first infusion device and the second infusion device comprises a physical connection at a first connection position of the set of connection positions.

Clause 12: The method of clause 11, wherein the recommended connection position is the same as the first connection position of the set of connection positions; and the indication confirms the coupling of the first infusion device and the second infusion device.

Clause 13: The method of clause 11, wherein the recommended connection position is different from the first connection position of the set of connection positions, and the indication indicates to couple the first infusion device and the second infusion device at the recommended connection position.

Clause 14: The method of clause 13, further comprising: detecting a decoupling of the first infusion device from the second infusion device; detecting a recoupling of the first infusion device to the second infusion device; determining a connection position of the first infusion device to the second infusion device based on the recoupling; comparing the connection position of the first infusion device to the second infusion device after the recoupling to the recommended connection position; and in response to determining the connection position of the first infusion device to the second infusion device after the recoupling is the same as the recommended connection position, removing the indication indicating to couple the first infusion device and the second infusion device at the recommended connection position.

Clause 15: The method of clause 14, further comprising outputting a connection confirmation indication.

Clause 16: The method of any one of clauses 14-15, further comprising in response to determining the connection position of the first infusion device to the second infusion device after the recoupling is the same as the recommended connection position, initiating a start operation of the first infusion device.

Clause 17: The method any one of clauses 1-16, wherein the first infusion device comprises an infusion pump.

Clause 18: The method any one of clauses 1-17, wherein the second infusion device comprises an infusion docking station.

Clause 19: The method any one of clauses 1-18, wherein the second infusion device comprises an infusion controller.

Clause 20: A method, comprising: detecting a coupling of a first infusion device and a second infusion device; determining a connection position of a set of connection positions associated with the second infusion device based on the coupling of the first infusion device and the second infusion device; and transmitting, to a remote device, a connection message including an identifier of the first infusion device; an identifier of the second infusion device; and an identifier of the connection position of the set of connection positions.

Clause 21: The method of clause 20, wherein the identifier of the connection position of the set of connection positions indicates a position of the coupling of the first infusion device relative to the second infusion device.

Clause 22: The method of any one of clauses 20-22, wherein the connection message further includes a usage score of each connection position in the set of connection positions.

Clause 23: The method of clause 22, further comprising generating a usage score of each connection position of the set of connection positions based on historical connection data for each connection position in the set of connection positions.

Clause 24: The method of clause 23, wherein the historical connection data comprises an attach and detach event of the second infusion device.

Clause 25: The method of clause 24, further comprising: starting a timer based on the coupling of the first infusion device and the second infusion device; detecting a decoupling of the first infusion device and the second infusion device; stopping the timer based on the decoupling of the first infusion device and the second infusion device; and determining the attach and detect event based on the coupling of the first infusion device and the second infusion device and the decoupling of the first infusion device and the second infusion device, and a time period based on the timer associated with the attach and detach event of the second infusion device.

Clause 26: The method of any one of clauses 23-24, wherein the historical connection data comprises a start and stop event of the second infusion device.

Clause 27: The method of clause 26, further comprising: initiating a start operation of the first infusion device; starting a timer based on the start operation of the first infusion device and the second infusion device; initiating a stop operation of the first infusion device; stopping the timer based on the stop operation of the first infusion device and the second infusion device; and determining the start and stop event of the second infusion device based on the start operation and the stop operation, and a time period based on the timer associated with the start and stop event.

Clause 28: The method of clause 27, further comprising: initiating a pause operation of the first infusion device; pausing the timer based on the pause operation of the first infusion device; initialing a resume operation of the first infusion device; and resuming the timer based on the resume operation of the first infusion device.

Clause 29: The method of any one of clauses 23-28, wherein the historical connection data comprises a time period since a previous maintenance event.

Clause 30: The method of any one of clauses 23-29, wherein the historical connection data comprises an active time period of the second infusion device.

Clause 31: The method of any one of clauses 23-30, further comprising generating a maintenance message based on the usage score of one or more connection positions in the set of connection positions.

Clause 32: The method of clause 31, wherein generating the maintenance message based on the usage score of one or more connection positions in the set of connection positions, comprises: comparing the usage score for each connection position in the set of connection position to an overuse threshold for the set of connection positions; and determining a maintenance event for at least one connection position of the set of connection positions based on the usage score of one or more connection positions in the set of connection positions satisfying the overuse threshold, wherein the maintenance message indicates an identifier of the at least one connection position of the set of connection positions.

Clause 33: The method any one of clauses 20-32, wherein the coupling of the first infusion device and the second infusion device comprises a physical connection at a first connection position of the set of connection positions.

Clause 34: The method any one of clauses 20-33, wherein the first infusion device comprises an infusion pump.

Clause 35: The method any one of clauses 20-34, wherein the second infusion device comprises an infusion docking station.

Clause 36: The method any one of clauses 20-35, wherein the second infusion device comprises an infusion controller.

Clause 37: An infusion system, comprising: an infusion pump configured to deliver fluid to a patient; and an infusion pump controller configured to perform a method in accordance with any one of Clauses 1-36.

Clause 38: A processing system, comprising: memory comprising computer-executable instructions; and one or more processors configured to execute the computer-executable instructions and cause the processing system to perform a method in accordance with any one of Clauses 1-36.

Clause 39: A processing system, comprising means for performing a method in accordance with any one of Clauses 1-36.

Clause 40: A non-transitory computer-readable medium storing program code for causing a processing system to perform the steps of any one of Clauses 1-36.

Clause 41: A computer program product embodied on a computer-readable storage medium comprising code for performing a method in accordance with any one of Clauses 1-36.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database, or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An infusion system, comprising:
an infusion pump configured to deliver fluid to a patient; and
an infusion pump controller configured to:
detect a coupling of a first infusion device and a second infusion device;
store data indicative of a coupling event based on detecting the coupling of the first infusion device and the second infusion device;
determine a recommended connection position of a set of connection positions associated with the second infusion device based on the coupling event; and
output an indication of the recommended connection position of the set of connection positions for the coupling of the first infusion device and the second infusion device.

2. The infusion system of claim 1, wherein to determine the recommended connection position of the set of connection positions based on the coupling event, the infusion pump controller is further configured to:
generate a usage score based on historical connection data for each connection position in the set of connection positions;
compare the usage score for each connection position in the set of connection positions to a respective usage threshold for each respective connection position of the set of connection positions; and
determine the recommended connection position based on the usage score of one or more connection positions in the set of connection positions satisfying the respective usage threshold.

3. The infusion system of claim 2, wherein, for each connection position in the set of connection positions, the historical connection data comprises a number of start and stop operations of the second infusion device while coupled at the connection position.

4. The infusion system of claim 2, wherein the historical connection data comprises a number of attach and detach events of the second infusion device.

5. The infusion system of claim 2, wherein the historical connection data comprises a time period since a previous maintenance event.

6. The infusion system of claim 2, wherein the historical connection data comprises an active time period of the second infusion device.

7. The infusion system of claim 1, wherein the recommended connection position of the set of connection positions is further based on one or more operational parameters of the first infusion device.

8. The infusion system of claim 1, wherein the infusion pump controller is further configured to detect a second coupling of the second infusion device and one or more additional infusion devices, wherein the recommended connection position is further based on the second coupling.

9. The infusion system of claim 1, wherein:
the coupling of the first infusion device and the second infusion device comprises a physical connection at a first connection position of the set of connection positions;
the recommended connection position is different from the first connection position of the set of connection positions;
the indication indicates to couple the first infusion device and the second infusion device at the recommended connection position; and
the infusion pump controller is further configured to:
detect a decoupling of the first infusion device from the second infusion device;
detect a recoupling of the first infusion device to the second infusion device;
determine a connection position of the first infusion device to the second infusion device based on the recoupling;
compare the connection position of the first infusion device to the second infusion device after the recoupling to the recommended connection position; and
in response to determining the connection position of the first infusion device to the second infusion device after the recoupling is the same as the recommended connection position, remove the indication indicating to couple the first infusion device and the second infusion device at the recommended connection position.

10. The infusion system of claim 9, wherein the infusion pump controller is further configured to, in response to determining the connection position of the first infusion device to the second infusion device after the recoupling is the same as the recommended connection position, initiate a start operation of the first infusion device.

11. An infusion system, comprising:
an infusion pump configured to deliver fluid to a patient; and
an infusion pump controller configured to:
detect a coupling of a first infusion device and a second infusion device;
determine a connection position of a set of connection positions associated with the second infusion device based on the coupling of the first infusion device and the second infusion device; and
transmit, to a remote device, a connection message including an identifier of the first infusion device; an identifier of the second infusion device; and an identifier of the connection position of the set of connection positions.

12. The infusion system of claim 11, wherein the identifier of the connection position of the set of connection positions indicates a position of the coupling of the first infusion device relative to the second infusion device.

13. The infusion system of claim 11, wherein the connection message further includes a usage score of each connection position in the set of connection positions.

14. The infusion system of claim 13, wherein the infusion pump controller is further configured to generate the usage score of each connection position based on historical connection data for each connection position in the set of connection positions.

15. The infusion system of claim 14, wherein:
the historical connection data comprises an attach and detach event of the second infusion device; and
the infusion pump controller is further configured to:
start a timer based on the coupling of the first infusion device and the second infusion device;
detect a decoupling of the first infusion device and the second infusion device;
stop the timer based on the decoupling of the first infusion device and the second infusion device; and
determine the attach and detect event based on the coupling of the first infusion device and the second infusion device and the decoupling of the first infusion device and the second infusion device, and a time period based on the timer associated with the attach and detach event of the second infusion device.

16. The infusion system of claim 14, wherein:
the historical connection data comprises a start and stop event of the second infusion device; and
the infusion pump controller is further configured to:
initiate a start operation of the first infusion device;
start a timer based on the start operation of the first infusion device and the second infusion device;
initiate a stop operation of the first infusion device;
stop the timer based on the stop operation of the first infusion device and the second infusion device; and
determine the start and stop event of the second infusion device based on the start operation and the stop operation, and a time period based on the timer associated with the start and stop event.

17. The infusion system of claim 16, wherein the historical connection data comprises a time period since a previous maintenance event.

18. The infusion system of claim 16, wherein the historical connection data comprises an active time period of the second infusion device.

19. The infusion system of claim 16, wherein the infusion pump controller is further configured to generate a maintenance message based on the usage score of one or more connection positions in the set of connection positions.

20. The infusion system of claim 19, wherein to generate the maintenance message based on the usage score of one or more connection positions in the set of connection positions, the infusion pump controller is further configured to:
compare the usage score for each connection position in the set of connection position to a respective overuse threshold for each respective connection position of the set of connection positions; and
determine a maintenance event for at least one connection position of the set of connection positions based on the usage score of one or more connection positions in the set of connection positions satisfying the respective overuse threshold, wherein the maintenance message indicates an identifier of the at least one connection position of the set of connection positions.
